(19) United States
(12) Patent Application Publication
(10) Pub. No.: US 2024/0295492 A1
(43) Pub. Date: Sep. 5, 2024

(54) DEVICE AND METHOD FOR DETERMINING A CONCENTRATION OF A MATERIAL IN A MEASUREMENT VOLUME

(71) Applicant: AVL List GmbH, Graz (AT)

(72) Inventors: Andreas Pein, Paldau (AT); Gerald Steiner, Graz (AT); Mario Schaller, Graz (AT)

(21) Appl. No.: 18/572,931

(22) PCT Filed: Jun. 24, 2022
(86) PCT No.: PCT/AT2022/060218

§ 371 Date: Dec. 21, 2023

(30) Foreign Application Priority Data
Jun. 25, 2021 (AT) A50532/2021

(51) Int. Cl. G01N 21/31; G01N 33/00

(52) U.S. Cl. CPC G01N 21/31; G01N 33/0027

(57) ABSTRACT

According to the invention, in order to allow reliable concentration measurements of a gaseous or solid material in a measurement volume, at least one part of an exhaust plume in the measurement volume is captured by an imaging unit and a total passage path of the light beam through the exhaust plume in the measurement volume is determined from the captured image of the at least one part of the exhaust plume, and a concentration of the gaseous or solid material in the measurement volume is determined from the determined decrease in the light intensity and from the total passage path.

The present invention relates to a device for determining the concentration of at least one gaseous or solid material in at least one measurement volume at a stationary measurement point, wherein at least one measurement unit is provided to emit a light beam with a predetermined light intensity through the measurement volume, and at least one detector is provided in order to detect the light beam after passage through the measurement volume, wherein the at least one detector is designed to determine a decrease in the light intensity due to the at least one gaseous or solid material in the measurement volume.

The invention further relates to a corresponding method for determining the concentration of at least one gaseous or solid material in an exhaust plume in at least one measurement volume.

Emissions of materials in exhaust gases, especially in individual traffic, are a versatile topic due to the increasing number of vehicles not only in the course of climate heating, but also in the course of the health stress for humans by nitrogen oxides, partially combusted fuel components and fine dust particles. Developments of the last decades aimed at avoiding the ejection of partially burned compounds on one side in the course of the catalytic converter installation in gasoline engines, and in the case of catalysts in diesel engines on the other hand to prevent the ejection of nitrogen oxides. The permissible values of emitted materials are often determined via national and supranational standards.

Nevertheless, there are still vehicles in use in public spaces today which, although they met the legal standards for exhaust gas reduction at the time of their registration, are considered to be high emitters when used over a longer period of time. The reason for this can be, for example, the lack of retrofitting of a catalytic converter or missing maintenance, if for example, in the case of a diesel catalyst, the refilling of urea is omitted and a proper function of an SCR (selective catalytic reduction) catalyst is no longer provided. Among other things, this can also be due to a lack of knowledge regarding the (non-)functionality of just these components during the journey.

Exhaust gas measurements are largely limited to systems which measure materials in the exhaust gas, such as gaseous materials or particles, in the vehicle itself, for example in or after the tailpipe. However, these systems are limited to a small number of test vehicles and can therefore not give a representative image of a plurality of different vehicles in real operation. Exhaust gas measurements within the framework of regular checking of the vehicle in a repair shop are also not representative because such checks are carried out only at large time intervals. It is therefore attempted to enable exhaust gas measurements from vehicles in real operation in public space. Remote Sensing, also in the sense of real driving emission (RDE) measurements, takes place at a stationary measurement point and can, for example, be attached to an advantageously pre-installed infrastructure, such as toll stations, street lights, bridges, or also building facades in the city and the like. This could be used, for example, to notify vehicle holders of vehicles with high emissions and/or to provide maintenance. However, when the devices are set up, care must be taken to ensure that representative results are obtained. In general, crossing regions with traffic lights and thus potential stoppage of the vehicles should be avoided. Furthermore, it has been shown that a slight slope of the road at the measurement point is suitable for generating a positive motor load.

In remote sensing, a light source is often used which emits a characteristic wavelength or wavelength range(s) in order to detect a gaseous material, such as carbon monoxide or nitrogen oxides. A detector enables, for example, a measurement of the attenuation of the light that is transmitted through the exhaust plume. However, it can also be provided to measure particles, such as soot particles, as a material. This can then be realized, for example, via light scattering or by measuring the attenuation of the back beam in relation to the radiated light.

However, the reliable measurement of such materials in exhaust plumes can lead to different difficulties. On the one hand, the emissions of materials from different motors or also other energy systems, such as fuel cells, are different and must be measurable with the same system. Furthermore, the concentration range to be measured fluctuates very strongly and is dependent on the vehicle class to be measured (e.g., truck vs motorcycle). Especially low concentrations cause problems during evaluation. Differences in the operating temperature of a motor can also result in differences in the materials to be measured.

Although absorption values can thus be determined, they are difficult to compare or not at all comparable due to the differences mentioned above. In particular, no concentration of a gaseous or solid material can thus be determined in the exhaust plume.

Today available systems in the prior art detect gaseous or solid materials as an optical mass (OM), which can generally be based on a constant reference concentration of a second material in the exhaust plume, for example carbon dioxide (CO₂). Such a reference value can supply values representing some exhaust clouds. For example, such a reference value can be used for exhaust clouds from gasoline combustion engines in order to reliably determine also different OMs of different materials of the exhaust clouds. If, for example, the absorption of a material differs in different exhaust plumes, the absorption of the reference value is proportional and can be set in proportion. Consequently, the concentration of the material can be calculated via the known concentration of the reference value and the absorption of the reference value. This type of calculation can function relatively reliably for internal combustion engines with gasoline, since the concentration value is relatively constant due to the stoichiometric combustion. However, this type of concentration calculation cannot be used for vehicles that run on diesel, as the non-stoichiometric combustion process can cause very strong fluctuations in the reference concentration, for example a CO₂ concentration. This procedure is therefore unreliable.

CN 103712929 A1 describes a system for localizing and measuring an exhaust cloud by means of an optical camera, infrared camera, and spectroscopic unit. The optical camera measures the vehicle speed and determines the license plate number. The infrared camera locates the position of the exhaust plume and the spectroscopic measurement unit is aligned in order to measure the exhaust plume. Although the disclosure improves the localization of the exhaust plume, it does not enable determining a concentration value of an exhaust gas component.

EP 3 789 755 discloses a measurement method for exhaust plumes by means of multispectral analysis with a plurality of infrared cameras. The IR cameras are arranged above or next to the road and can measure, for example, carbon monoxide and/or carbon dioxide by means of suitable filters. The arrangement has the disadvantage that no spectroscopic evaluation is used and therefore the possibility of measurements of exhaust gas components is limited. Furthermore, it has the disadvantage that such methods have very low sensitivities and are furthermore not able to measure absolute concentrations directly.

It is therefore an object of the present invention to enable reliable concentration measurements of a gaseous or solid material in a measurement volume in remote sensing measurement.

According to the invention, this object is achieved by a device mentioned at the outset in that an image unit is provided in the device in order to capture at least a portion of the exhaust plume when an exhaust plume is partially or completely present in the measurement volume, that an evaluation unit is provided in the device in order to determine an overall passage path of the light beam through the exhaust plume in the measurement volume from at least one captured image of the at least one portion of the exhaust plume, and that the evaluation unit is configured to determine a concentration of the gaseous or solid material in the measurement volume from the determined decrease in light intensity and the total passage path.

The concentration determination according to the invention from the total passage path of the light beam through an exhaust plume in the measurement volume and from a determined decrease in the light intensity of the light beam due to the at least one gaseous or solid material allows the concentration of the material to be determined directly, reliably and accurately. This determination of the concentration is in particular also independent of the expansion or composition of the exhaust plume.

In an advantageous embodiment, the at least one measurement unit is arranged at a distance from at least one first reflection unit, wherein the measurement volume is formed between the at least one measurement unit and the at least one first reflection unit, and the at least one measurement unit is provided to emit a light beam in the direction of the at least one reflection unit, and wherein the at least one first reflection unit is provided to reflect the light beam after passing through the measurement volume and to transmit it to at least one detector. Such a design can simplify the arrangement of the detector in the device because the detector no longer necessarily has to be arranged opposite to the measurement unit. The use of the device is thus more flexible. In this case, it is particularly advantageous if the light beam reflected at the reflection unit passes through the measurement volume at least once more before reaching the detector. Due to the double passage of the light beam through the measurement volume, the sensitivity of the concentration measurement can be increased, and lower concentrations of the gaseous or solid material can be measured.

The total passage path is favorably the sum of the partial passage paths of all passages of the light beam through the exhaust plume.

In one variant of the invention, a multiplexer unit is provided in the measurement unit, which multiplexer unit generates a plurality of light beams, and the measurement unit emits the plurality of light beams at different locations through the measurement volume, and at least one detector detects each of the plurality of light beams, preferably after a reflection at a reflection unit. A spatially resolved concentration measurement in the measurement volume can thereby be achieved in a simple manner. The concentration of the gaseous or solid material can thus be determined at different locations of an exhaust plume in the measurement volume.

If at least two of the plurality of light beams are guided in the measurement unit via an optical path in each case, wherein the optical paths preferably have different optical path lengths at least in part, the detection of these light beams at a detector can be simplified because the light beams arrive at the detector in a time-staggered manner due to the resulting different transit times.

In order to achieve a two-dimensional resolution of the measurement volume, in one variant of the invention, the measurement unit radiates at least one first light beam of the plurality of light beams in a first direction through the measurement volume, and the measurement unit radiates a second light beam of the plurality of light beams in a second direction through the measurement volume, wherein the first direction is different from the second direction. The concentration of the gaseous or solid material can thus be detected in different dimensions. If this is combined with a spatially resolved concentration measurement, a very precise reconstruction of a concentration distribution in the exhaust plume is possible.

The number of passages of the light beam through the measurement volume can be further increased if a second reflection unit is provided in the device, which second reflection unit is arranged at a distance and opposite from the at least one first reflection unit, and the measurement unit radiates the light beam in an angle deviating from a normal to a first reflection plane of the first reflection unit, wherein the first reflection unit reflects the light beam emitted by the measurement unit to the opposite second reflection unit and the opposite second reflection unit reflects the light beam back to the first reflection unit, wherein the at least one detector is provided to detect the light beam after a plurality of such reflections. Particularly advantageously, the angle of the light beam can be adjustable in order to be able to select and adjust the number of reflections application-related. In such a variant, a positioning optical unit is provided in the measurement unit, with which the angle of the light beam can be adjusted.

A plurality of measurement units can also be provided in the device, wherein at least two of the measurement units emit a light beam through different measurement volumes. In a further variant, a plurality of measurement units is provided in the device, wherein at least two of the measurement units emit a light beam with different directions through the same measurement volume. The device can thus be adapted very flexibly and easily to different applications.

The image unit preferably has at least one camera and/or at least one lidar unit.

To protect sensitive reflection units from dirt or damage, a protective film is advantageously arranged over the reflective unit so that it can be replaced. In this way, necessary maintenance intervals can be reduced.

The object of the invention is also achieved by a method mentioned at the outset, in which the at least one measurement unit radiates a light beam with a predefined light intensity through the exhaust plume in the measurement volume, the light beam is detected by a detector after passing through the exhaust plume, and the detector determines a decrease in the light intensity of the light beam due to the at least one gaseous or solid material, wherein according to the invention at least a portion of the exhaust plume is captured by an image unit in the measurement volume and an total passage path of the light beam through the exhaust plume in the measurement volume is determined from the captured image of the at least one portion of the exhaust plume, and that a concentration of the gaseous or solid material in the measurement volume is determined from the determined decrease in light intensity and the total passage path.

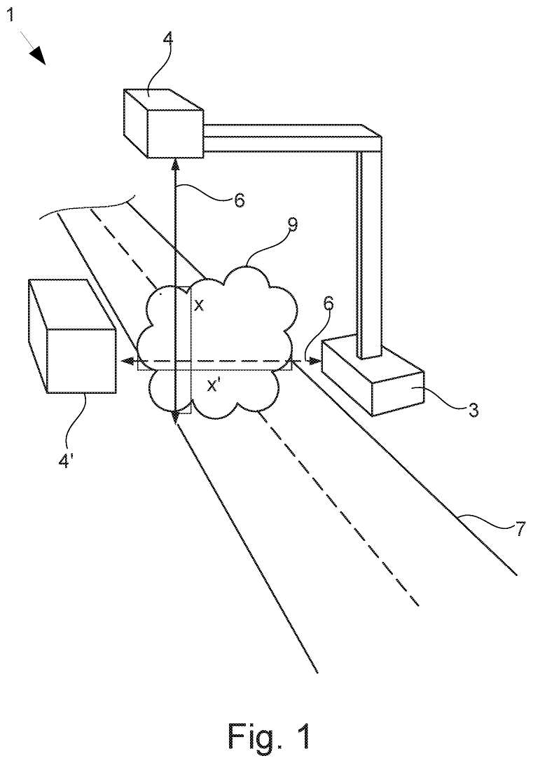

The present invention is described in greater detail below with reference to FIGS. 1 through 6, which show schematic and non-limiting advantageous embodiments of the invention by way of example. In the figures:

FIG. 1 shows the principle of the remote exhaust gas measurement (remote sensing),

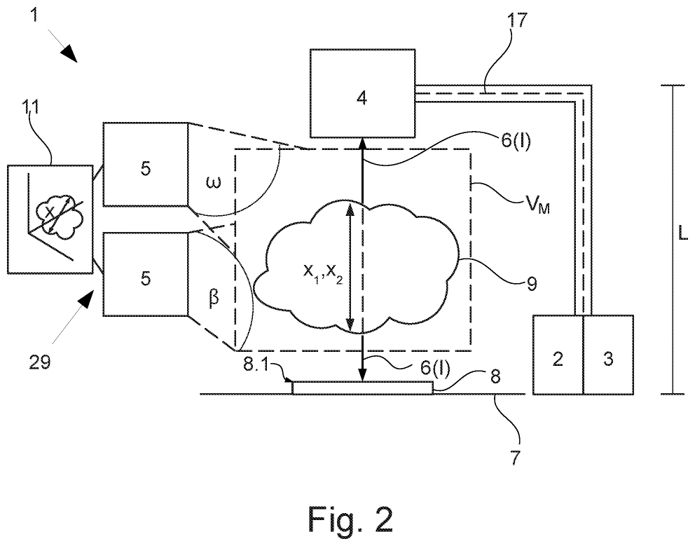

FIG. 2 shows an embodiment according to the invention of the concentration determination in an exhaust plume,

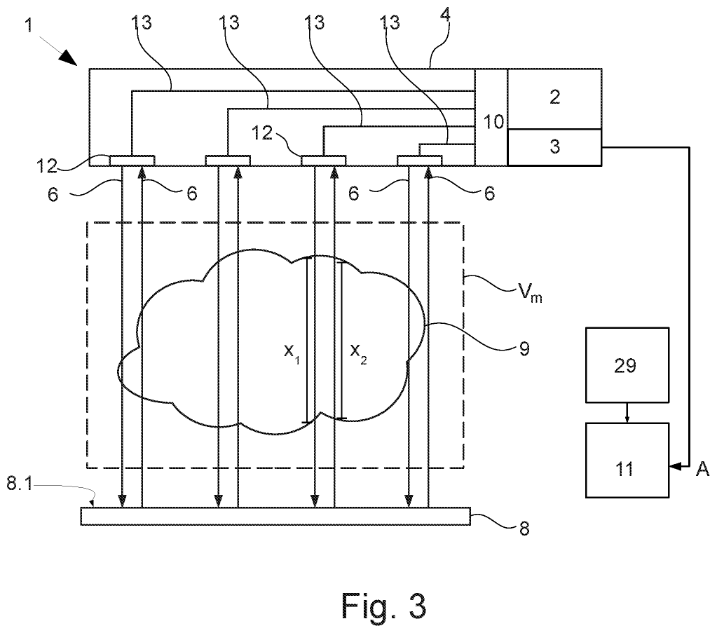

FIG. 3 shows an advantageous embodiment of a measurement unit,

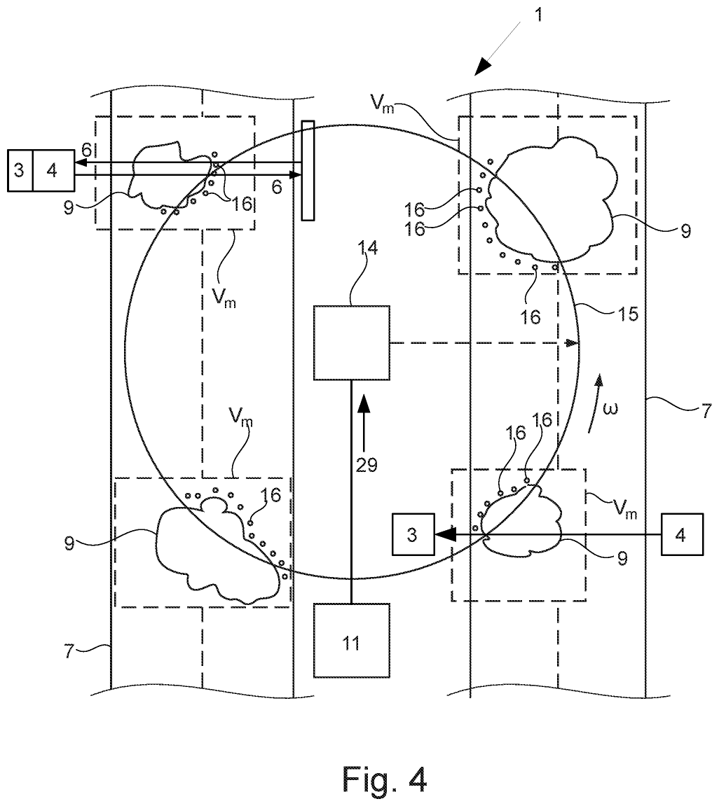

FIG. 4 shows an embodiment of the device having a lidar unit as an image unit,

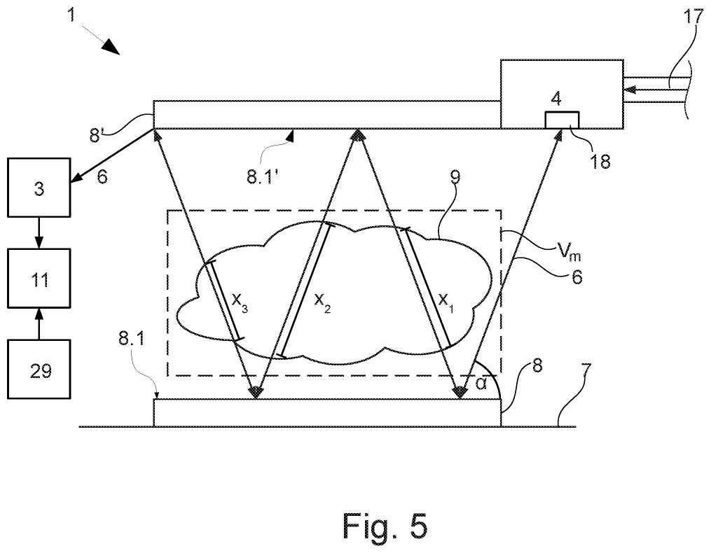

FIG. 5 shows an embodiment of the device with a multiple passage of the light beam through the exhaust plume, and

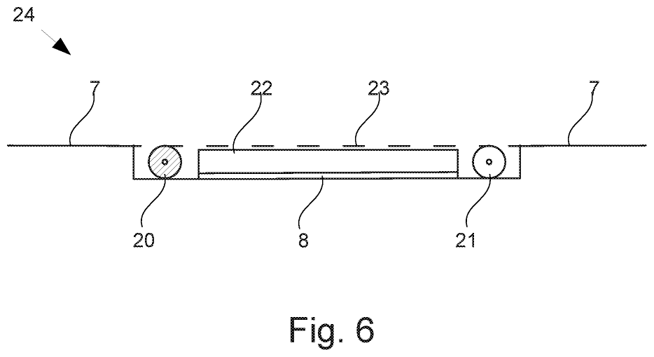

FIG. 6 shows an embodiment for the protection of a reflection unit.

FIG. 1 shows a device 1 according to the prior art for measuring a gaseous or solid material in an exhaust plume 9 which is ejected from an emission source, such as a vehicle in the public space.

A wide variety of gaseous and solid (e.g., particles) components can occur in the exhaust plume 9. The materials in the exhaust plume 9 can originate from any type of emission source, for example on a surface 7, for example from a vehicle such as a passenger car (passenger car), truck, but also a single-track vehicle such as a motorcycle, motorized bicycle, and the like, which have an internal combustion engine. Emissions of a different emission source, such as fuel cells, which usually emit only water vapor and no pollutants, can also be measured via such a device 1. The detection of such emission sources can be helpful in determining, for example, the proportion of vehicles having low or high emission values in road traffic. The measurement can take place on a surface 7, for example a road, advantageously at a certain distance above a surface 7. However, it is also conceivable that the device 1 is arranged on the side of an exhaust plume 9 and the measurement takes place parallel to a surface 7, or the device 1 can also be installed in the surface 7 itself. Combinations of measurements from a plurality of sides are also conceivable.

The device 1 can measure an exhaust plume 9, for example, also at other locations away from a surface 7. It is conceivable that the device 1 measures an exhaust cloud 9 of an aircraft when starting or landing on a landing list on an airport. It is also conceivable for an exhaust plume 9 of a ship to be measured, for example in a harbor basin or in a lock.

In addition to application in the automobile industry, other applications in which exhaust plumes 9 with gaseous or solid materials are formed as emissions are also conceivable, for example in the process industry. For example, emissions can be measured in chimneys that can have a diameter of several meters. The measurement volume V_{M} would be formed in the chimney.

The invention is not limited to the above-mentioned applications, but rather all possible uses that become apparent to the person skilled in the art are conceivable. However, the exhaust plume 9 does not necessarily have to originate from a vehicle, but can in principle originate from any emission source. An example is an exhaust cloud from an industrial process which is emitted, for example, on a chimney.

The materials in an exhaust plume 9 can be gaseous materials, such as carbon dioxide (CO₂), carbon monoxide (CO), nitrogen oxides (NO_{X}), sulfur dioxide (SO₂), gaseous polycyclic aromatic hydrocarbons (PAH) and the like. However, it is also conceivable to measure solid materials in an exhaust plume 9, such as soot particles. The materials and their concentrations in the exhaust plume 9 of a vehicle are usually dependent on the fuel type, the internal combustion engine, the operating state of the internal combustion engine, and the status of a catalytic converter or exhaust gas aftertreatment system (if present). For example, an internal combustion engine which is not yet at operating temperature often emits a higher concentration of partially burned materials, such as polycyclic aromatic hydrocarbons, than at normal operating temperature. Likewise, different materials are emitted in different operating states (e.g. given by current rotational speed and current torque).

According to the invention, the concentration of such a gaseous or solid material in the exhaust plume 9 is to be measured.

Depending on the arrangement of the measurement unit 4 of the device 1, a light beam 6 can pass through the exhaust plume 9 on different passage paths x. The light beam 6 is detected and evaluated after passing through the exhaust plume 9 in a detector 3. For example, in different arrangements, as given in FIG. 1, the passage path x of the light beam 6, which is normal to the surface 7, can be different from another passage path x′ of a different light beam 6, which runs parallel to the surface 7, and can therefore be dependent on the arrangement of the measurement units 4, 4′ relative to the exhaust plume 9. This can lead to different measurement results for the material in the exhaust plume 9 for the nominally identical concentration of a material, because absorption is generally higher at a longer passage path x, x′.

FIG. 2 shows an embodiment of the device 1 according to the invention, with which a concentration of a gaseous or solid material in an exhaust plume 9 can be reliably measured. In this embodiment, at least one light source 2 is provided in the device 1, which light source generates light with a predefined light intensity. The light source 2 can emit, for example, monochromatic light, for example a laser light which has a defined wavelength with a predefined light intensity. In particular, quanta cascade lasers (QCL) can be used, but other types and combinations of light sources and lasers are also conceivable in order to cover different wavelength ranges. It is also conceivable that the light source 2 has a polychromatic emitting lamp, such as a lamp in the ultraviolet (UV) or also in the infrared range (IR). A monochromator in the light source 2 is also conceivable in order to select wavelengths in a targeted manner. The light source 2 generates a primary light beam 17 with a specific light intensity and at least one wavelength. The primary light beam 17 can, for example, be guided to at least one measurement unit 4 via a light guide, such as a fiber optic cable, a mirror system or another suitable optical system. However, the light source 2 can also be integrated in the measurement unit 4 and generate the primary light beam 17 in the measurement unit 4.

The measurement unit 4 generates a light beam 6 from the primary light beam 17 of the light source 2 with at least one defined wavelength and a defined light intensity I which is emitted by the measurement unit 4. Preferably, the wavelength and the light intensity I of the light beam 6 correspond to the wavelength and the light intensity of the primary light beam 17. However, the measurement unit can also change the light properties of the primary light beam 17 (e.g. wavelength or light intensity), for example by means of a monochromator or a beam splitter in order to generate the light beam 6. The light beam 6 can also be generated in a pulsed manner, for example in the form of individual light packets.

To carry out the concentration measurement, the measurement unit 4 radiates the light beam 6 directly or indirectly in the direction of the measurement volume V_{M} so that the light beam 6 passes through the measurement volume V_{M} and through an exhaust plume 9 partially or completely therein. The light beam 6 is detected by a detector 3 after passing through the exhaust plume 9 in the measurement volume V_{M}.

In the embodiment of the invention according to FIG. 2, the measurement unit 4 is arranged at a distance L from a reflection unit 8 with a reflection plane 8.1, for example on the surface 7. The reflection plane 8.1 of the reflection unit 8 is arranged facing the measurement unit 4. The measurement volume V_{M} is formed between the measurement unit 4 and the reflection unit 8, in which an exhaust plume 9 to be measured can be partially or completely located when the device 1 is used as intended. An incident light beam is reflected at the reflection unit 8 or at the reflection plane 8.1.

The distance L can be dependent on the application, for example dependent on the vehicles passing through, but also on the direction of the measurement. The distance L can be lower, for example, if a measurement is carried out parallel to the surface 7, and can be higher when measured normal to the surface 7. In any case, the distance L can be selected to match the application.

The orientation and alignment of the reflection unit 8 can be chosen as desired and in an application-related manner. The reflection unit 8 can be mounted, for example, parallel to the plane of the surface 7, normal to the plane of the surface 7, or also at any angle to the plane of the surface 7. Advantageously, the reflection unit 8 can also be integrated in a surface 7 and can be protected by means of a suitable coating or installation against damage and contamination, for example caused by vehicles which travel over the surface 7.

The arrangement of the reflection unit 8 and the orientation of the light beam 6 emitted by the measurement unit 4 is in any case selected such that the light beam 6 passes through the measurement volume V_{M} and impinges on the reflection unit 8. However, the light beam 6 does not have to be directed directly onto the reflection unit 8, but can also be directed in the direction of the reflection unit 8 via an optical system, for example a mirror arrangement.

The exact dimensions of the measurement volume V_{M} can be specified or can be selected by a user and can be, for example, dependent on the expected extent of an exhaust plume 9. The measurement volume V_{M} can, for example, also be selected smaller than an expected exhaust plume 9 in order to prevent mixing of different exhaust clouds 9 from different emission sources in a measurement volume V_{M} .

In order to carry out the concentration measurement with a device 1 in the embodiment according to FIG. 2, the measurement unit 4 radiates the light beam 6 directly or indirectly in the direction of the reflection unit 8. The emitted light beam 6 passes through the measurement volume V_{M} and is reflected at the reflection unit 8. The reflected light beam 6 is guided to at least one detector 3 which detects the reflected light beam 6. The reflected light beam 6 can thereby pass through the measurement volume V_{M} a second time, depending on the design of the reflection. The light beam 6 thus passes through the measurement volume V_{M} and an exhaust plume 9 partially or completely therein at last once. Preferably, the reflected light beam 6 is reflected at the reflection unit 8 such that the reflected light beam 6 also passes through the measurement volume V_{M} and through an exhaust plume 9 located therein. Due to the repeated passing through of the exhaust plume 9 by the light beam 6, the sensitivity of the concentration measurement can be increased, because the light along the beam path is affected several times from the gaseous or solid materials in the measurement volume V_{M} and thus more significant measurement signals are possible.

The detector 3 can be arranged in spatial proximity to the light source 2, for example in a common housing with the light source 2, but can also be arranged, for example, in the measurement unit 4 or at any other location of the device 1. In the device 1 according to FIG. 2, the detector 3 is installed with the light source 2 in a housing and the reflected light beam 6 is guided along the same optical path as the primary light beam 17.

According to the invention, the detector 3 measures an attenuation of the light intensity I of the detected light beam 6 due to a gaseous or solid material in the measurement volume V_{M}. This attenuation of the light intensity I can be related to a reference measurement, which is carried out in the absence of substances in the measurement volume V_{M} and results in a reference light intensity I₀. This can be used, for example, to calculate an absorption 1−(I/I₀), referred to as A for short, or transmission I/I₀, of a specific wavelength, or even several different wavelengths. The transmission and the absorption A can be converted into one another and can be regarded as equivalent.

For solid materials in measurement volume V_{M} light intensity loss can be caused by light scattering, such as forward or sideways scattering. The detector 3 can also detect, for example, light scattering on solid particles in the measurement volume V_{M} and therefore measure a light intensity decrease due to scattering. The size of solid particles in the detector 3 can thus also be estimated. It is also possible to capture a spectrum over different wavelengths, and to carry out a spectroscopic evaluation, for example in the form of multivariate analyses or fingerprinting of a material. This means that several different materials can be measured at the same time. This can also be enabled via suitable optical filters or via monochromators.

The decrease in the light intensity I of the light beam 6 detected by the detector 3 depends on the total passage path x of the light beam 6 through an exhaust plume 9 in the measurement volume V_{M} and can also vary depending on the arrangement of the measurement unit 4 and/or the reflection unit 8. Different sizes of the exhaust plume 9, depending on the vehicle, can also lead to different measured values at the detector 3.

The measurement of an absorption of a gaseous or solid material is known to be frequency-dependent and should therefore take place at or near the absorption maximum in order to obtain a reliable result. For example, CO₂ has characteristic vibrational oscillations at a wave number (reciprocal of the wavelength) of 1388 cm¹ (asymmetric stretching oscillation) and at 667 cm¹ (bending oscillation). According to the Lambert-Beer law, the absorption A is dependent on the total passage path x, the concentration c and an absorption coefficient k (as known material parameter). This relationship can be given with the formula

$$ln \frac{I}{{I}_{0}} = k * x * c$$

In order to determine a reliable concentration c of a material, knowledge of the total passage path x and the absorption A is therefore required. Absorption A (or, equivalently, a decrease in light intensity) can be determined via detector 3. However, the total passage path x is dependent on the extent of the exhaust plume 9 in the measurement volume V_{M} and the beam path of the light through the exhaust plume 9 and is usually not known.

For a reliable concentration measurement of a wide variety of exhaust plumes 9, it is therefore advantageous to have knowledge about the total passage path x of the light through the exhaust plume 9, since this does not have to rely on the above-mentioned calculation via an optical mass with the listed limitations. The total passage path x depends on the beam path of the light beam 6 and thus on how often and at which point the light beam 6 passes through the exhaust plume 9.

In order to detect the total passage path x, according to the invention an image unit 29 is provided in the device 1 in order to image at least a portion of the measurement volume VM, preferably from different directions (e.g. angles ω, β). The image unit 29 generates at least one image of a portion of an exhaust plume 9 in the measurement volume VM, preferably a plurality of images from different directions, which is processed in an evaluation unit 11. The evaluation unit 11 can now determine from the obtained at least one image the total passage path x of the light beam 6 through the exhaust plume 9. For this purpose, the evaluation unit 11 can reconstruct at least part of an image of an exhaust plume 9 in the measurement volume VM. The total passage path x through the exhaust plume 9 can be determined from the image of the part of the exhaust plume 9 using the known beam path of the light (which is determined by the arrangement of the reflection unit 8 and the orientation of the light beam 6) through the measurement volume VM.

The total passage path x comprises at least the partial passage path x1 of the light beam 6 through the exhaust plume 9. If the light beam 6 is reflected and send through the exhaust plume 9 once more, then the partial passage distance x2 of the reflected light beam 6 is also added. The total passage path x is thus the sum of the individual partial passage paths of the light beam 6 through the exhaust plume 9.

For example, the known dimensions of the measurement volume V_{M} can be used to calculate the dimensions of at least one reconstructed part of the exhaust plume 9 in the measurement volume V_{M} and thus a partial passage path x1, x2.

In an arrangement as in FIG. 2, in which the light beam 6 is directed substantially normal to the reflection plane 8.1 of the reflection unit 8, the partial passage paths x1, x2 of the emitted light beam 6 and of the reflected light beam 6 are the same because the light beam 6 is reflected in the opposite direction. It can therefore be sufficient to determine and duplicate only the partial passage path x1 of the emitted light beam 6 or the partial passage path x2 of the reflected light beam 6 in order to obtain the total passage path x.

To determine the partial passage paths x1, x2, or the total passage path x derived therefrom, a 2D projection of the exhaust plume 9 in the plane of the light beam 6 and/or of the reflected light beam 6 can be generated from the images captured with the image unit 29, and the partial passage paths x1, x2 can thus be determined directly. Advantageously, the evaluation unit 11 creates a spatial reconstruction of the exhaust plume 9, for which purpose images of the exhaust plume 9 are required from different directions. This reconstruction can also be dependent, for example, on a running variable, such as the time. For example, a time-dependent expansion of an exhaust plume 9 can thus be determined.

In an advantageous embodiment, the evaluation unit 11 can obtain data about outside temperature and atmospheric humidity. Depending on the outside temperature and air humidity, differences in the evaluation and reconstruction of an exhaust plume 9 can occur. For example, temperature differences in the summer between the surroundings and the exhaust plume 9 are less pronounced than in winter. This can lead to a determined partial passage path x1, x2 having differences depending on the season. In order to avoid or at least limit this error source, a correction factor can be provided for calculating the reconstruction depending on the outside temperature and the air humidity. The evaluation unit 11 can thus carry out a more reliable calculation of the total passage path x independently of the ambient conditions.

The evaluation unit 11, usually a computer with corresponding evaluation software, and can also obtain data for absorption A (or the light intensity of the detected light beam 6) from the at least one detector 3. A reference light intensity 10 can be stored in the evaluation unit 11 and can be assumed to be known. The evaluation unit 11 uses the total passage path x and the decrease in the light intensity, or an absorption A, in order to determine the concentration c of a gaseous or solid material, for example according to the above-mentioned Lambert-Beer law. In an advantageous embodiment, different absorptions A at different wavelengths of the light beam 6 can also be used in order to determine concentrations c of different gaseous or solid materials. This can take place one after the other at one wavelength in each case or also in the form of spectroscopic evaluation at a plurality of wavelengths at the same time.

The image unit 29 can be designed in the form of at least one camera 5, preferably a plurality of cameras 5 (as shown in FIG. 2). An embodiment of the image unit 29 with one or more lidar units, one or more radar units or combinations of such units or with cameras is also conceivable. In addition, further embodiments of an image unit 29 are possible.

The image unit 29, for example the at least one camera 5, can be arranged, for example, on the measurement unit 4. In one variant, the image unit 29 is installed on a separate device, or existing infrastructure in the region of the device 1, such as bridges, houses, street lights, or the like, is used for arrangement. With a plurality of measurement volumes V_{M} the image unit 29 can also be arranged such that it images, for example, a plurality of measurement volumes V_{M} at the same time. The number of required image units 29 can thus be kept low.

In one embodiment of the image unit 29 with a plurality of cameras 5, these are installed at different locations in order to image the measurement volume V_{M} from different directions ω, β. When captureing the measurement volume V_{M} from different directions, the reconstruction of the exhaust plume 9 can be facilitated or improved.

The at least one camera 5 can capture images of the measurement volume V_{M} and thus also an exhaust plume 9 present in the measurement volume V_{M}. However, it is also possible that the camera 5 additionally captures metadata of a vehicle, such as size, type, or also the license plate. When using a camera 5 as an image unit 29, available image processing software can be used, for example, in order to reconstruct the exhaust cloud 9 or a portion thereof from the images.

The at least one camera 5 can be, for example, an infrared camera which captures thermal images of the present exhaust plume in the measurement volume V_{M}. The heat distribution in the exhaust plume 9 can thus also be detected, which can have an influence on the gaseous or solid materials or the absorption coefficients k. Due to the temperature differences, convection and diffusion phenomena can occur which cause a distribution of materials over time. Individual concentrations c of materials can also be dependent on the temperature, since some reactions proceed only at a higher temperature or are temperature-dependent.

However, the at least one camera 5 can also operate in the ultraviolet (UV) or visible (VIS) region, for example, or also in both regions (UV/VIS cameras). UV or VIS is higher-energy radiation than IR and excites electron transition in molecules and can be more advantageous for measurement.

In one embodiment, the at least one camera 5 can be designed as a multi-and hyperspectral camera. In this case, instead of the classic simple image in a simple spectral range, a high number of spectral bands are used. This can be advantageous in order to detect a significantly higher color quality and color differences, since each pixel already contains a complete color spectrum. For example, the snapshot mosaic technique is used in such a camera 5.

The measurement point, in particular the measurement volume V_{M}, is stationary during the measurement with the device 1, and therefore a stationary measurement is realized with the device 1 at a specific stationary measurement point. This means that the exhaust plume 9 can move or change relative to the measurement volume V_{M} during the measurement, but the device 1, specifically the individual units of the device 1, remain stationary at the measurement point. In particular, the at least one light source 2, a measurement unit 4, a detector 3, a reflection unit 8 remain stationary during the measurement. If further units are present in the device 1, such as a multiplexer unit 10, these are also stationary during the measurement.

The device 1 according to the invention is thus in particular not a measurement unit which is installed in a moving vehicle for exhaust gas measurement and moves along with the vehicle during the measurement.

However, the device 1 is necessarily arranged in a stationary manner only during the measurement. The device 1 or parts thereof can also be moved between two measurements. In the course of remote sensing measurements, it may, for example, be necessary for the device 1 to change between different measurement points, for example in order to detect a larger part of vehicles traveling in a larger area (e.g., a city). In this context, it can also be provided to mount the device 1 partially or completely on a trailer in order to be able to switch the device 1 between the measurement points in a mobile manner. The main part of the device 1, in particular a light source 2, a measurement unit 4, a detector 3, and an image unit 29, can be preassembled on an extendable frame on the trailer. Parts of the device 1, such as the reflection unit 8, which can be applied at the measurement point, are then arranged in a suitable manner at the measurement point. However, all units are again stationary during the measurement.

It can happen that different exhaust plumes 9 of emission sources located behind or next to one another, such as vehicles, mix in the measurement volume V_{M}. The measurement can then be adapted accordingly, for example via positioning or alignment of the image unit 29, in particular of the at least one camera 5.

In one variant, a plurality of measurement units 4 is provided in the device 1 according to the invention, also at different distances L from a respectively associated reflection unit 8. A plurality of reflection units 8 can also be provided, for example for each measurement unit 4 a separate or in each case one for different groups of measurement units. A plurality of measurement units 4 are advantageous for spatially resolved measurements of an exhaust plume 9 in a measurement volume V_{M} or also for a plurality of exhaust plumes 9 in different measurement volumes V_{M} at the same time. For this purpose, a primary light beam 17 of a light source 2 can also be correspondingly divided with optical systems, for example by means of beam splitters, deflection units, mirrors, multiplexers, etc., in order to supply light to each of the measurement units 4. However, a light source 2 for generating a primary light beam 17 can also be provided in each measurement unit 4, or in some thereof.

In a multiple measurement of an exhaust plume 9, an average value of the concentration of a gaseous or solid material in the exhaust plume 9 can be determined. However, it is also possible to determine a spatially resolved measurement of the concentration in the exhaust plume 9. With a corresponding arrangement of the measurement units 4 and reflecting units 8, a two-dimensional spatial resolution of the concentration of a gaseous or solid material in the exhaust plume 9 can also be determined. For this purpose, for example, a first group of light beams 6 could pass through the exhaust plume in a first direction and a second group of light beams 6 could pass through in a second, different direction, for example normal to the first direction.

FIG. 3 shows an advantageous embodiment of the measurement unit 4 with multiple measurement of an exhaust plume 9. In this embodiment, light source 2 and detector 3 are arranged in the measurement unit 4. In one variant, the light source 2 and/or the detector 3 can also be arranged externally of the measurement unit 4 at any suitable location. The image unit 29 is only indicated in FIG. 3 and can be designed as explained above in connection with FIG. 2 or also as explained below in connection with FIG. 4. The image unit 29 generates the images of the measurement volume VM which are processed in the evaluation unit 11. A multiplexer unit 10 is provided in the measurement unit 4, which multiplexer unit generates a plurality of light beams 6 (wherein only some are denoted in FIG. 3 with of the reference signs). The multiplexer unit 10 generates the plurality of light beams 6 from the primary light beam 17 (not shown in FIG. 3) of the light source 2.

The multiplexer unit 10 can be realized, for example, by an optical fiber multiplexer, optical encoder or optical splitter. A realization of the multiplexer unit 10 as a fiber optic switching network is also conceivable. Various embodiments of a multiplexer unit 10 are sufficiently known and the person skilled in the art can select a suitable for the particular application.

The individual emitted light beams 6 are guided to different locations or regions of the measurement volume V_{M}, whereby the light beams 6 pass through the exhaust plume 9 present in the measurement volume V_{M} at different points. The exhaust plume 9 can thus be detected in a spatially resolved manner. The individual light beams 6 can be detected by at least one detector 3 or reflected at at least one reflection unit 8 (as described in FIG. 3) as described above and directed as reflected light beams 6 to at least one detector 3 (which is arranged in the embodiment of FIG. 3 in the measurement unit 4) and thus can be detected.

Of course, a reflection unit 8 can be provided for each light beam 6 or a group of light beams 6. Equally, a detector 3 can be provided for each reflected light beam 6 or for a group of reflected light beams 6.

As described, the at least one detector 3 can now determine a decrease in the light intensity or an absorption A for each detected light beam 6 and transmit it to the evaluation unit 11. In the same way, the evaluation unit 11 can determine a total passage path x from the at least one image of the measurement volume V_{M} captured by the imaging unit 29 for each light beam 6 and, if applicable, the associated reflected light beam 6 (if it passes through the exhaust plume 9), as described. A concentration c of a gaseous or solid material can thus be determined at different points in the exhaust plume 9.

It is obvious that a two-dimensional spatial resolution of the exhaust plume 9 is also possible here if the device 1 is designed such that at least two groups of light beams 6 pass through the exhaust cloud 9 in different directions (preferably normal to one another). If such a two-dimensional resolution is carried out several times in time, a three-dimensional concentration distribution (in the case of a moving exhaust plume 9) or a time profile of the concentration distribution in an exhaust plume 9 can also be determined therefrom.

In the embodiment according to FIG. 3, the individual light beams 6 are guided in the measurement unit 4 via optical paths 13, for example optical waveguides, at the ends of which the light beams 6 are emitted via an emitting unit 12. If at least two of these optical paths 13 are designed with different optical path lengths, then the return beams 6 associated with the optical paths with different path lengths arrive at a detector 3 due to the different propagation times of the light at different points in time. This can simplify or facilitate the detection of different light beams 6 with an individual detector 3.

Of course, a plurality of measurement units 4 with multiplexer units 10, as described with reference to FIG. 3, can also be provided in the device 1. This again enables multiple measurements in one measurement volume V_{M}, preferably in order to achieve a two-dimensional resolution of the measurement volume V_{M}, or also the measurement of different measurement volumes V_{M}.

A spatially resolved determination of the concentration c in an exhaust plume 9 (for example with a measurement unit according to FIG. 3 or with a plurality of measurement units 4 as in FIG. 2 or also a combination of such arrangements) can then be used, for example, to simulate a continuous distribution of a gaseous or solid material in the exhaust plume 9, for example data driven or by means of hybrid modeling. Hybrid modeling is understood to mean a combination of discrete terms, such as, for example, the measured concentration values, and continuous terms, for example fluid/gas dynamics in an exhaust plume 9.

FIG. 4 shows a further embodiment of the device 1 according to the invention. In this embodiment, a plurality of measurement units 4 (in FIG. 4 are two thereof indicated for reasons of clarity) which can be designed as described above and for which one detector 3 is provided in each case. The plurality of measurement units 4 are used to carry out concentration measurements of a gaseous or solid material in an exhaust plume 9 in different measurement volumes V_{M}. The measurement volumes V_{M} can be different in volume, wherein the volumes depend on the measuring arrangement of the measurement units 4. For reasons of clarity, the beam paths for measuring the concentration are not shown completely or are only partially indicated in FIG. 4. In the embodiment of FIG. 4, an image unit 29 is provided which captures images from all exhaust clouds 9 in the different measurement volumes V_{M} and sends them to an evaluation unit 11. The evaluation unit 11 naturally also receives values of the absorptions A determined with detectors 3.

In this embodiment of the device 1, the image unit 29 is designed as a lidar unit 14. The lidar unit 14 can be arranged locally in the vicinity of the measurement units 4, but can also be arranged, for example, at a certain height above the measurement units 4, or can also be controlled in a mobile manner by means of a drone in order to carry out a measuring campaign.

A very accurate measurement of an exhaust plume 9 can be carried out with a lidar unit 14, wherein the lidar unit 14 captures images of at least a portion of the exhaust plume 9 in order to determine the total passage path x as described above.

The lidar unit 14 is based on a laser, for example a YAG laser with 1064 nm or 532 nm wave length, or similar embodiments, which are suitable for a person skilled in the art. IR lasers can also be used, although sufficient shielding can be necessary in order to avoid eye damage.

However, a lidar unit 14 in the UV or NIR region can also be used, for example, to measure gaseous or solid materials directly. Lidar is used inter alia in order to detect, in atmospheric measurements, for example, carbon dioxide (CO₂), sulfur dioxide (SO₂) and methane (CH₄). This can be used to carry out, for example, coarse estimations of gaseous or solid materials in the measured exhaust plume 9 or also to obtain a concentration measurement that is redundant with respect to the concentration measurement according to the invention.

The lidar unit 14 can move along at least one axis or can be pivoted about at least one axis in order to emit the laser light in different directions, so that an image of the surroundings and a possibly present exhaust plume 9 can be captured. In the embodiment according to FIG. 4, the lidar unit 14 is used in order to image different exhaust clouds 9 in different measurement volumes V_{M}. For this purpose, the lidar unit 14 scans the surroundings 15 (shown in FIG. 4 by a circle around the lidar unit 14) and, depending on the reflection time of the emitted laser pulse, images of the surroundings 15 are made. In FIG. 4, the mode of operation of the lidar unit 14 is shown in two dimensions. The lidar unit 14 rotates about an axis running through the lidar unit 14 and normal to the drawing plane of FIG. 4, and scans the surroundings 15 at a circular frequency ω.

In this case, portions 16 of the exhaust plumes 9 are detected, combined to form images of the exhaust plume 9, and can thus be reconstructed by the evaluation unit 11 to form a portion of an image of the exhaust plume 9.

However, such a lidar unit 14 can of course also be used for a single measurement volume V_{M}, for example as in FIG. 2 or 3. Equally, a plurality of lidar units 14 can be present in the device 1, which enable a more accurate and possibly larger image of the exhaust plume 9.

Combinations of lidar units 14 and cameras 5 are also conceivable. For example, concentrations of gaseous materials can thus be measured via the lidar unit 14, while a concentration of a solid material in the exhaust plume 9 can be detected via a measurement unit 4 according to the invention. A representative concentration measurement of a plurality of critical gaseous and solid materials of the exhaust plume 9 can thus take place.

FIG. 5 shows a further advantageous embodiment of the invention. In this embodiment, two reflection units 8, 8′ arranged at a distance from one another are provided. The corresponding reflection planes 8.1, 8.1′ of the two reflection units 8, 8′ are arranged facing one another. The light beam 6 is now emitted by the measurement unit 4 at an angle a which deviates from a normal to the first reflection plane 8.1 of the first reflection unit 8. The light beam 6 is reflected at the first reflection plane 8.1 and directed to the second reflection plane 8.1′ of the second reflection unit 8′. A further reflection of the light beam 6 back to the first reflection unit 8 takes place on the second reflection plane 8.1′. The light beam 6 thus runs back and forth in a plurality of reflections between the first reflection unit 8 and the second reflection unit 8′ and moves along a longitudinal extension of the reflection planes 8.1, 8.1′. A plurality of reflections can thus be achieved depending on the angle a. After the last reflection at the end of the reflecting units 8, 8′, the light beam 6 in the embodiment according to FIG. 5 is directed to a detector 3 which detects the light beam 6.

After the last reflection at the end of the mirroring units 8, 8′, the light beam 6 can, however, also be deflected in an embodiment (not shown), for example by a deflection mirror, and runs back in a number of further reflections between the first reflection unit 8 and the second reflection unit 8′ along a longitudinal extension of the reflection planes 8.1, 8.1′. In this case, the detector 3 can be arranged in the measurement unit 4. However, the detector 3 can in principle be arranged at any suitable location of the device 1 which makes it possible to detect the light beam 6 after a plurality of reflections of the light between the first reflection unit 8 and the second reflection unit 8′.

Due to the multiple reflections, there is a plurality n of passages of the light beam 6 through an exhaust plume 9 in the measurement volume V_{M}. As described above, the resulting partial passage paths xn of the light through the exhaust plume 9 can be determined with the image unit 29, which in turn results in the total passage path x as the sum of the partial passage paths xn (here n=1, 2, 3). This in turn allows the concentration determination of at least one gaseous or solid material in the exhaust plume 9.

The sensitivity of the concentration measurement can be further increased by the plurality n of passages of the light beam 6 through the exhaust plume 9, and very low concentrations of a gaseous or solid material can also be determined in the exhaust plume 9.

In order to be able to adjust the plurality n of passages of the light beam 6 through the exhaust plume 9 selectively and as required, a positioning optical unit 18 can also be provided in the measurement unit 4, which positioning optical unit allows the angle α of the emitted light beam 6 to be adjusted. Such a positioning optical unit 18 can be, for example, an x-y galvanometer or also an adjustable mirror.

A further advantageous embodiment for a protection of a reflection unit 8 is a protective film unit 24, which is shown in FIG. 6. This is used to arrange a protective film 23 replaceably over a reflection unit 8 in order to protect it from soiling or damage (e.g. from scratches). The protective foil 23 is designed to be sufficiently transparent. A soiled protective film 23 can be replaced if necessary by a clean protective film 23. According to FIG. 6, a possible embodiment of a protective film unit 24 consists of a first roller 20 on which clean protective film 23 is wound. Clean protective film 23 can be unwound from this first roller 20 and arranged above a reflection unit 8. A second roller 21 can be provided on which the soiled protective film 23 can be wound. When used as intended, clean protective film 23 is unwound from the first roller 20, if necessary, and soiled protective film 23 is simultaneously wound up by the second roller 21. In this advantageous embodiment, the reflection unit 8 is arranged below the surface 7.

The unwound protective film 23 is arranged above the reflection unit 8 in order to protect the reflection unit 8 from contamination or damage. For stability reasons, a mechanical protection 22 can also be provided between the reflection unit 8 and the protective film 23, which should have sufficient optical permeability. One of the two rollers 20, 21 can be driven in order to bring about a movement of the protective film 23 over the reflection unit 8 as required. An automation unit can also be provided for this purpose, which controls the drive of the driven roller 20, 21. Advantageously, the rollers 20, 21 can be driven when a limit value is undershot, for example a light intensity loss of a light beam 6 detected with a detector 3. The automation unit can then automatically control the driven roller 20, 21 in order to move the protective film 23. The soiled protective film 23 above the reflection unit 8 can thus be easily changed and, if necessary, replaced by an uncontaminated. This can be advantageous if the reflection unit 8 is generally exposed to high contamination.

A heating device, for example an electric heating, can also be integrated into the protective film unit 24, preferably in the mechanical protection 22. The heating device can prevent the optical systems of the device 1 from being impaired by the formation of ice or puddles on the surface of the protective film 23 during wetness, such as rain, mist, snow, for example.

1. A device for determining the concentration of at least one gaseous or solid material in at least one measurement volume at a stationary measurement point, wherein at least one measurement unit is provided to emit a light beam with a predetermined light intensity through the measurement volume, and at least one detector is provided to detect the light beam after it has passed through the measurement volume, wherein the at least one detector is configured to determine a decrease in the light intensity due to the at least one gaseous or solid material in the measurement volume, wherein an image unit is provided in the device, in order to capture at least a part of an exhaust plume in the case of partial or complete presence of an exhaust plume in the measurement volume, in that an evaluation unit is provided in the device in order to determine a total passage path of the light beam through the exhaust plume in the measurement volume from at least one captured image of the at least one part of the exhaust plume, and in that the evaluation unit is configured to determine a concentration of the gaseous or solid material in the measurement volume from the determined decrease in the light intensity and the total passage path.

2. The device according to claim 1, wherein the at least one measurement unit is arranged at a distance from at least one first reflection unit, the measuring volume being formed between the at least one measurement unit and the at least one first reflection unit, and the at least one measurement unit is configured to emit a light beam in the direction of the at least one reflection unit, and in that the at least one first reflection unit is provided to reflect the light beam after it has passed through the measurement volume and to transmit it to at least one detector.

3. The device according to claim 2, wherein the light beam reflected at the reflection unit passes through the measurement volume (V_{M}) at least once more before reaching the at least one detector.

4. The device according to claim 3, wherein the total passage path is the sum of the partial passage paths of all passages of the light beam through the exhaust plume.

5. The device according to claim 1, wherein characterized in that a multiplexer unit is provided in the measurement unit, which multiplexer unit generates a plurality of light beams, and the measurement unit emits the plurality of light beams at different locations through the measurement volume and at least one detector detects each of the plurality of light beams, preferably after a reflection at a reflection unit.

6. The device according to claim 5, wherein at least two of the plurality of light beams are guided in the measurement unit via an optical path in each case, wherein the optical paths preferably have different optical path lengths at least in part.

7. The device according to claim 5, wherein characterized in that the measuring unit emits at least one first light beam of the plurality of light beams in a first direction through the measuring volume and the measuring unit emits a second light beam of the plurality of light beams in a second direction through the measuring volume, the first direction being different from the second direction.

8. The device according to claim 2, wherein characterized in that a second reflection unit is provided in the device, which is arranged at a distance from and opposite the at least one first reflection unit and the measurement unit emits the light beam at an angle deviating from a normal to a first reflection plane of the first reflection unit, wherein the first reflection unit reflects the light beam emitted by the measurement unit to the opposite second reflection unit and the opposite second reflection unit reflects the light beam back to the first reflection unit, wherein the at least one detector is set up to detect the light beam after a plurality of such reflections.

9. The device according to claim 8, wherein a positioning optical unit is provided in the measurement unit, with which the angle of the light beam is adjustable.

10. The device according to claim 1, wherein in the device, a plurality of measurement units are provided, wherein at least two of the measurement units emit a light beam through different measurement volumes.

11. The device according to claim 1, wherein a plurality of measurement units are provided in the device, wherein at least two of the measurement units emit a light beam with different directions through the same measurement volume .

12. The device according to claim 1, wherein the image unit is selected from the group consisting of a camera and a lidar unit comprises at least one camera and/or at least one lidar unit.

13. The device according to claim 2, wherein a protective foil is arranged replaceably above the at least one reflection unit.

14. A method for determining the concentration of at least one gaseous or solid material in an exhaust plume in at least one measuring volume at a stationary measuring point, wherein the at least one measuring unit emits a light beam with a predetermined light intensity (I) through the exhaust plume in the measuring volume, the light beam is detected by a detector after passing through the exhaust plume and the detector determines a decrease in the light intensity of the light beam due to the at least one gaseous or solid material, wherein at least a part of the exhaust plume in the measuring volume is captured by an imaging unit and a total passage path of the light beam through the exhaust plume in the measuring volume is determined from the captured image of the at least one part of the exhaust plume, and in that a concentration of the gaseous or solid material in the measuring volume is determined from the determined decrease in light intensity and the total passage path.
